(19) United States
(12) Patent Application Publication
(10) Pub. No.: US 2017/0342217 A1
(43) Pub. Date: Nov. 30, 2017

(54) ANTIBODY

(71) Applicant: NATIONAL CHIAO TUNG UNIVERSITY, Hsinchu City (TW)

(72) Inventors: Yaw-Kuen LI, Hsinchu City (TW); Mo-Yuan SHEN, Taoyuan City (TW); Hsiu-Pen LIN, Chiayi City (TW)

(21) Appl. No.: 15/674,514

(22) Filed: Aug. 10, 2017

Related U.S. Application Data
(62) Division of application No. 15/006,135, filed on Jan. 26, 2016.
(60) Provisional application No. 62/108,034, filed on Jan. 26, 2015.

(51) Int. Cl. C08G 83/00; C08G 73/02; A61K 31/704; A61K 47/62; A61K 47/59; A61K 47/56; G01N 33/58; C07K 16/32

(52) U.S. Cl. CPC C08G 83/004; G01N 33/582; C08G 73/028; C07K 16/32; A61K 47/62; A61K 47/595; A61K 47/56; A61K 31/704

(57) ABSTRACT

An antibody includes a polyamidoamine (PAMAM) dendrimer and a first functional group. The polyamidoamine (PAMAM) dendrimer includes a plurality of branches and each of the branches has a phenylboronic acid (PBA) terminal group. The first functional group is bonded to at least one of the PBA terminal groups.

### RELATED APPLICATIONS

The application is a Divisional Application of the U.S. application Ser. No. 15/006,135, filed Jan. 26, 2016, which claims priority to U.S. Provisional Application Ser. No. 62/108,034, filed Jan. 26, 2015, all of which are herein incorporated by reference.

### BACKGROUND

### Technical Field

The present invention relates to a dendrimer, dendrimers having functional groups and antibodies comprising the dendrimers having functional groups.

### Description of Related Art

Dendrimer is a kind of polymer that has branches extending from the core as tree branches. Dendrimer is mainly defined by three components: a core, a layer of the branches extending from the core and exterior terminal groups. Dendrimer can be synthesized by using a divergent method or a convergent method. Because of the unique nature of dendrimer, it causes much attention in related fields. It mainly includes the following aspects: regular structure, relatively controllable molecular weight, a large number of surface terminal groups, and highly geometric symmetry. The exterior terminal groups of dendrimer provide many surface modifications and application possibilities to enhance the ability or resolve the problems in drug targeting and immunostaining.

### SUMMARY

In accordance with embodiments of the present invention, a dendrimer of dendrimer of Formula (I) is provided:

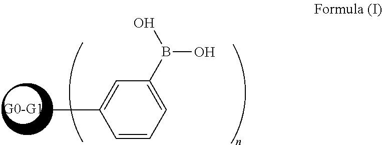

wherein G0-G10 represent generation-0 to generation-10 dendrimers; the dendrimers comprise a plurality of branches ;each branch comprises terminal groups

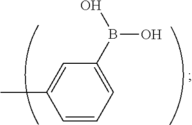

and n is an integer from 4-4096.

In some embodiments, the dendrimer is polyamidoamine (PAMAM) dendrimer.

In accordance with embodiments of the present invention, a dendrimer having functional groups includes a PAMAM dendrimer comprising a plurality of branches and each of the branch having a phenylboronic acid (PBA) terminal group; and a plurality of first functional groups bonded to at least part of the PBA terminal groups.

In some embodiments, each of the first functional groups comprise a drug group, a fluorescent group, a peptide group or a dopamine derivative group.

In some embodiments, each of the first functional groups comprises a drug group, and the drug group is doxorubicin (DOX).

In some embodiments, each of the first functional groups comprises a fluorescent group, and the fluorescent group comprises a fluorescein isothiocyanate (FITC) group or a Cyanine dye group.

In some embodiments, each of the first functional groups comprises a peptide group and the peptide group has ability to recognize an epidermal growth factor receptor (EGFR).

In some embodiments, each of the first functional groups comprises a group of

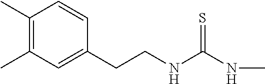

In some embodiments, the dendrimer having functional groups further includes a plurality of second functional groups, wherein each of the first functional groups comprise a peptide group having ability to recognize antigen, and each of the second functional groups comprise a drug group or a fluorescent group.

In accordance with embodiments of the present invention, an antibody includes the foregoing dendrimer having functional groups.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion.

FIGS. 1A-1C illustrate the preparation of dendrimers loaded with fluorescent molecules or drugs, in accordance with some embodiments.

FIG. 2A illustrates reaction of dendrimers loaded with fluorescent molecules and modified by antibodies, in accordance with some embodiments.

FIG. 2B is a photo of fluorescein samples of the present invention in comparison with conventional method.

FIG. 2C is an immuno-staining image and a bright field image of Serratia marcescens labeled by dendrimers loaded with fluorescent molecules and modified by antibodies, in accordance with one embodiment of the present invention.

FIG. 2D is an immuno-staining image and a bright field image of Streptococcus pneumoniae labeled by dendrimers loaded with fluorescent molecules and modified by antibodies, in accordance with one embodiment of the present invention.

FIGS. 3A-3C are photos of fluorescent intensity of dendrimers loaded with Cy5 fluorescent molecules and modified by antibodies, in accordance with one embodiment of the present invention.

FIG. 3D is a immuno-staining image of Streptococcus pneumoniae labeled by dendrimers loaded with Cy5 fluorescent molecules and modified by antibodies, in accordance with one embodiment of the present invention.

FIG. 3E is the bright-field image of FIG. 3D.

FIG. 3F is the superimposed image of FIG. 3D and 3E.

FIG. 4 is a photo of fluorescent intensity of PBA-dendrimers loaded with doxorubicin and modified by antibodies, in accordance with one embodiment of the present invention.

### DETAILED DESCRIPTION

The following disclosure will discuss the way to use and manufacture the embodiments. However, it should be recognized that the present invention provides innovative concept in practice, which can be presented by wide variety of specific contents. The following discussion is intended to be illustrative and is not intended to limit the scope of the present invention.

The following disclosure provides many different embodiments, or examples, for implementing different features of the provided subject matter. Specific examples of components and arrangements are described below to simplify the present disclosure. These are, of course, merely examples and are not intended to be limiting.

Embodiments relate to biological modified dendrimers having high loading efficiency of fluorescent dyes and drugs and the manufacturing method thereof are provided, which describe the formation of intermediate stages and various embodiments of biological modified dendrimers.

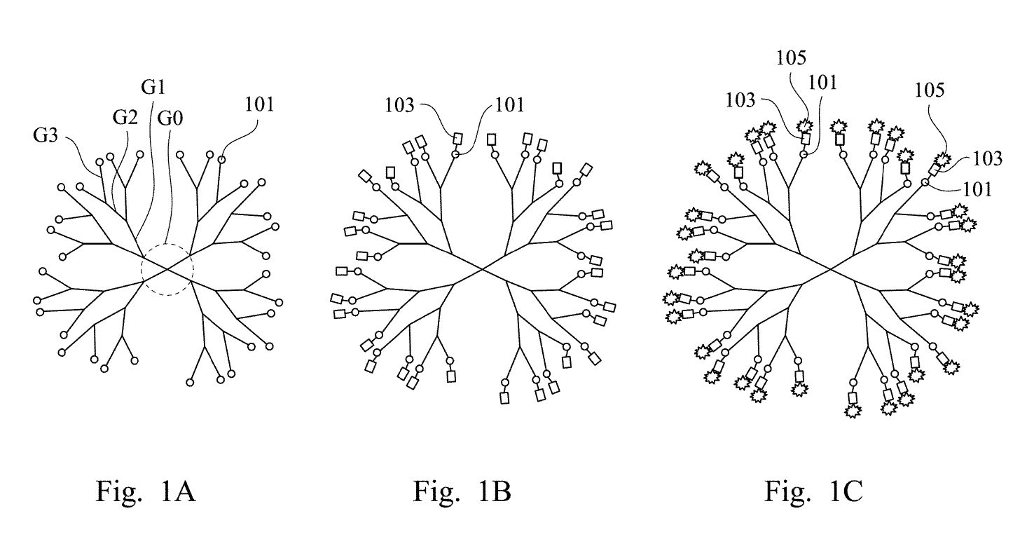

FIGS. 1A-1C illustrate the preparation of dendrimers loaded with fluorescent molecules or drugs, in accordance with some embodiments. First, a dendrimers is provided as shown in FIG. 1A. Dendrimers can be, for example, several generation branched polyamidoamine (PAMAM) dendrimers. Specifically, generation-0 (G0) is the central core that has four branches. Two branches will extend from each branch of generation-0 to form eight branches of generation-1. Again, two branches extend from each branch of generation-1 to form six-teen branches of generation-2. Similarly, two branches extend from each branch of generation-2 to form thirty-two branches of generation-3, and so on. The dendrimer illustrated in FIG. 1A is only for exemplary embodiment, other dendrimers still apply to the present invention. In accordance with the embodiments of the present invention, dendrimers can be generation-0, generation-1, generation-2, generation-3, generation-4, generation-5, generation-6, generation-7, generation-8, generation-9 or generation-10 dendrimers. In some embodiments, the outer branches of dendrimers connect to groups 101. In some embodiments, the groups 101 are —NH2 group.

Then, as shown in FIG. 1B, after dendrimers react with isothiocyanatophenylboronic acid, phenylboronic acid (PBA) terminal groups 103 are formed on the groups 101. Different generation dendrimers have different branch quantities. For example, dendrimers that have phenylboronic acid terminal groups can be the dendrimers represented by the following Formula (II).

The following describes the embodiment of forming dendrimers of formula (II). First, 1 equivalent G-3 PAMAM dendrimer in methanol solution is mixed with 1 mL tetrahydrofuran (THF) solution. The THF solution contains 32 equivalents 4-isothiocyanate phenylboronic acid and 32 equivalents trimethylamine. The mixed solution is stirred vigorously at room temperature for 48 hours. Then, the reaction is monitored by thin layer chromatography (TLC) and visualized by using ninhydrin stain. 2mL ether is then added into the solution to yield the product as white precipitant. The white precipitant is the compound represented by Formula (II). The product is further collected by centrifugation.

As shown in FIG. 1C, dendrimers with PBA terminal groups then load functional groups 105. In accordance with the embodiments of the present inventions, the functional groups can be, for example, fluorescent dyes and/or drugs.

In some embodiments, dopamine can react and couple with drugs or fluorescent dyes to form dopamine derivatives, which can react and couple with PBA-dendrimers to obtain dendrimers with functional groups 105. In other embodiments, dopamine react with other compound first to obtain the dopamine derivatives of Formula (III):

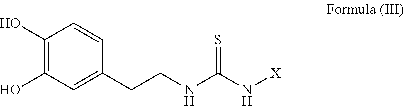

Group X can be —COOH, —S-SR, —SH, —N₃, —NCS, —C≡CH or maleimide; R is an alkyl group. However, as one of ordinary skill in the art will recognize, these process conditions are only intended to be illustrative, not to limit the scope of the embodiments.

In another embodiment, isothiocyanato dopamine reacts with nitrilotriacetic acid (NTA) to form dopamine derivatives, as shown in the following Reaction (I). The dopamine derivatives then react with the terminal groups of PBA-dendrimers.

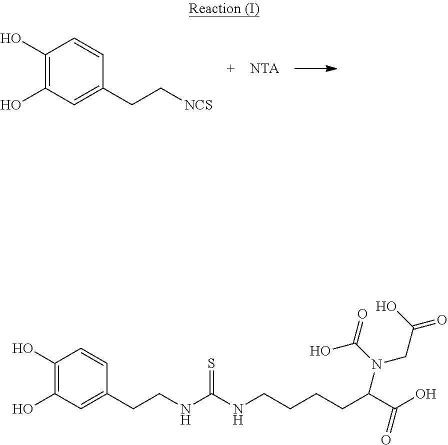

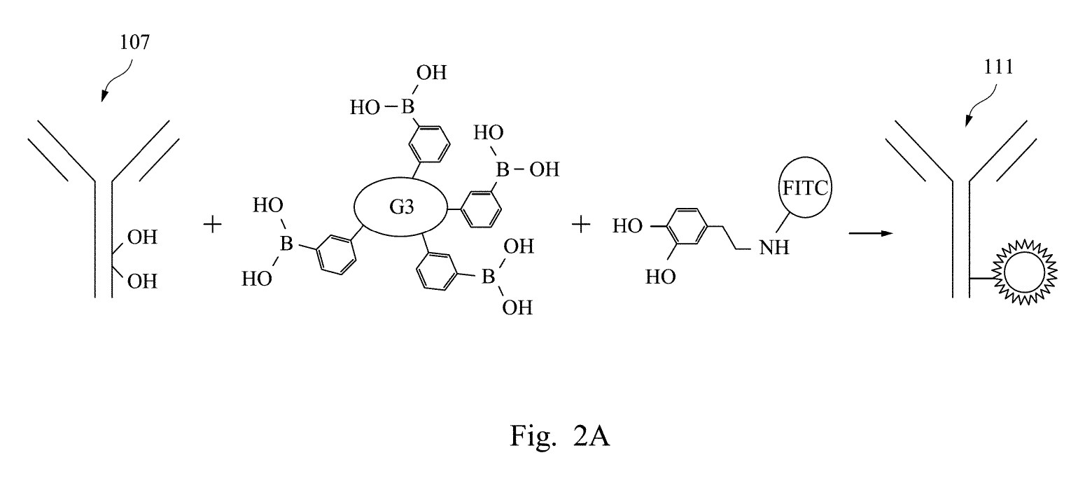

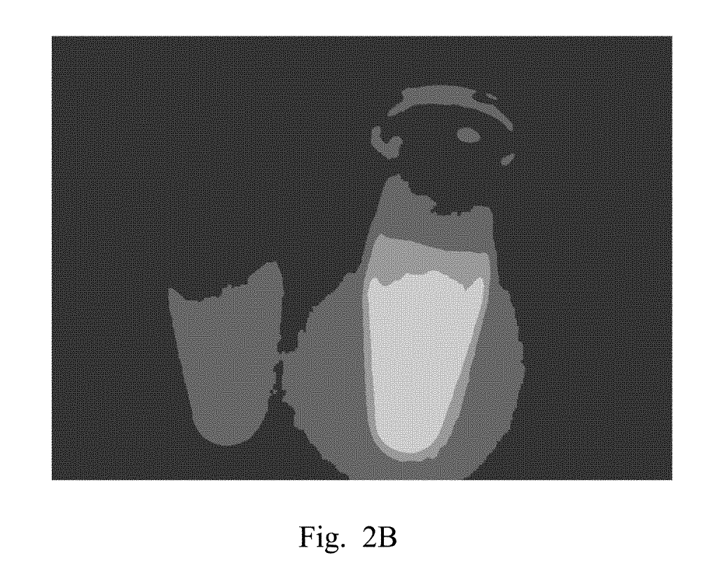

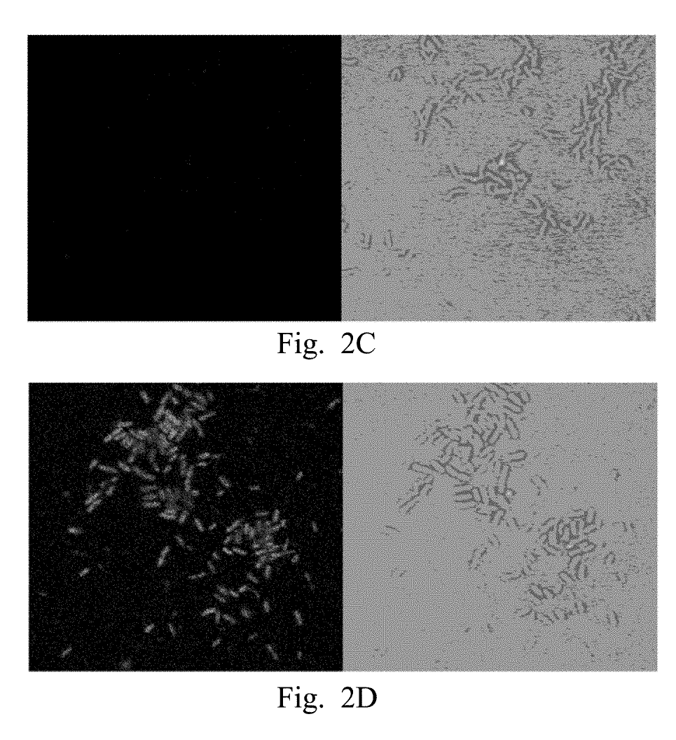

FIG. 2A illustrates reaction of dendrimers loaded with fluorescent molecules and modified by antibodies, in accordance with some embodiments. In one embodiment, dopamine reacts with fluorescent dye fluorescein isothiocyanate (FITC) to form dopamine derivatives:

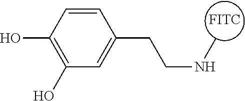

The dopamine derivatives (dopamine-FITC) react with dendrimers having PBA terminal groups to load the dendrimers with fluorescent groups. The dendrimers loaded with fluorescent groups then conjugate to antibodies 107 to obtain antibody-modified and fluorescein-labeled dendrimers 111 while possessing antigen recognition ability. In one embodiment, one equivalent FITC react with one equivalent dopamine. After trimethylamine (TEA) and dimethylformamide (DMF) are added to react at room temperature for 1 hour, the product dopamine-FITC is obtained, shown as the following Reaction (2):

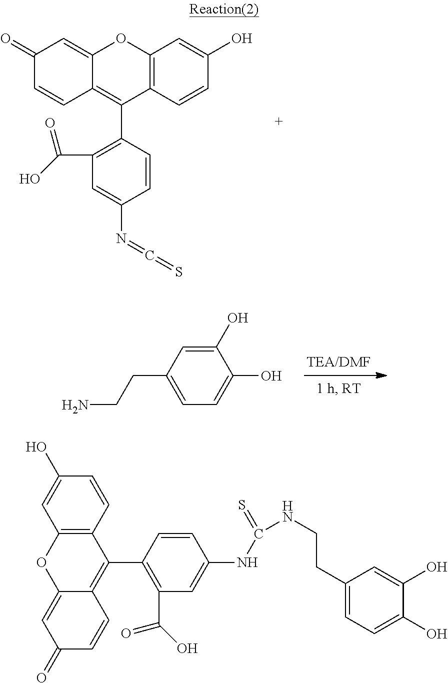 [SMILES: O=C(O)c1cc(N=C=S)ccc1-c1c2ccc(=O)cc-2oc2cc(O)ccc12 ; NCCc1ccc(O)c(O)c1 ; O=C(O)c1cc(NC(=S)NCCc2ccc(O)c(O)c2)ccc1-c1c2ccc(=O)cc-2oc2cc(O)ccc12]

In accordance with various embodiments of the present invention, PBA-dendrimers can couple with dopamine-derivatives before or after couple with glycoproteins such as antibodies. In some embodiments, PBA-dendrimers can couple with recombinant proteins (e.g. His-tag, Halo-tag proteins), peptides or saccharides.

In some embodiments, antibodies can react with PBA-G3 dendrimers, then react with dopamine-FITC. In other embodiments, antibodies, PBA-G3 dendrimers and dopamine-FITC can react at the same time by calculating the appropriate equivalents of the dopamine-FITC. Dendrimers with PBA terminal groups, for example PBA-G3 dendrimers, can react with glycol moiety of antibodies. Part of PBA terminal groups couple with glycol moiety while the rest of the PBA terminal groups couple with dopamine-FITC. Each antibody can couple with two PBA-G3 dendrimers loaded with FITC fluorescent molecules. In one embodiment, 50 μL antibody solution (0.91 mg/mL in phosphate buffer solution, PBS) is mixed with PBA-G3 dendrimers at 4° C. for 16 hours. Dopamine-FITC in PBS solution (125 μL, 2.3 mM) is added to the solution of antibody and PBA-G3 dendrimers, after the reaction is finished, the solution is under oscillation for 1 hour. The solution is dialyzed and 1000-fold diluted with PBS at 4° C. The procedure is repeated 3 times, each time for 2 hours. After purification, the modified antibodies (antibodies-dendrimer-FITC) are quantitative analyzed by UV-vis spectrophotometer. The result shows that every antibody conjugates 28+/−2 fluorescent groups in average.

Conventional approach is to directly conjugate FITC fluorescent molecules to antibodies, but FITC will react with any —NH2 groups and bond to them, thus it is possible to impact bonding of antibodies and antigen recognition ability. In prior art, the number of FITC fluorescent molecules that a single antibody can conjugate to varies, it is typically 3 to 5 in better condition. In accordance with some embodiments of the present invention, 28+/−2 fluorescent groups conjugate to every antibody in average to sharply enhance fluorescent intensity without affecting the recognition ability of antibody.

FIG. 2B is a photo of fluorescein samples of the present invention in comparison with conventional method. The sample in the left side of FIG. 2B uses conventional N-Hydroxysuccinimide (NHS)/1-Ethyl-3 -(3 -dimethylaminopropyl)carbodiimide (EDC) method. The sample in the right side of FIG. 2B uses the dendrimers provided by the present invention and loaded with fluorescent molecules and modified by antibodies. It is clear that the fluorescent intensity of the present invention is much stronger.

FIG. 2C is an immuno-staining image and a bright field image of Serratia marcescens labeled by dendrimers loaded with fluorescent molecules and modified by antibodies, in accordance with one embodiment of the present invention. FIG. 2D is an immuno-staining image and a bright field image of Streptococcus pneumoniae labeled by dendrimers loaded with fluorescent molecules and modified by antibodies, in accordance with one embodiment of the present invention. In accordance with embodiments of FIG. 2C and FIG. 2D, antibodies that can recognize Streptococcus pneumoniae is used, thus FIG. 2C is the control group and FIG. 2D is the test group. As shown in FIG. 2C and FIG. 2D, there is no fluorescence in the left picture of FIG. 2C while there is strong fluorescence in the left picture of FIG. 2D. Thus in accordance with embodiments of the present invention that dendrimers loaded with fluorescent molecules will not cause any unfavorable effects on the recognition ability of antibody.

In some embodiments, cyanine dye (Cy) can be used as another type of fluorescent dye, for example, Cy2, Cy3, Cy3.5, Cy5, Cy5.5 and Cy7. In one embodiment, Cy5 is used to react with dopamine-NCS to prepare dopamine-Cy5, as shown in the following Reaction (3).

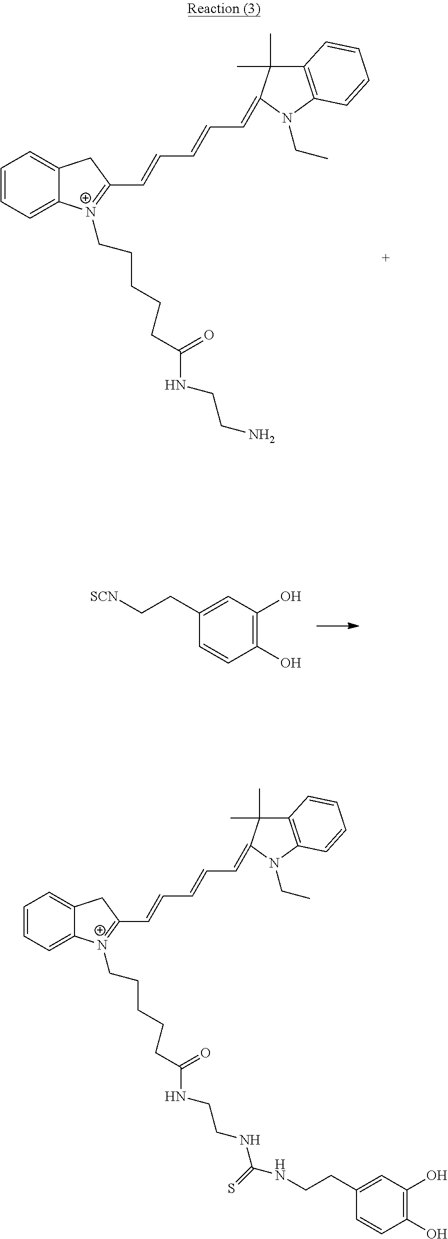

In some embodiments, PBA-G3 dendrimers can react with glycol moiety of the antibodies. Part of the terminal groups couple with glycol moiety while the rest can couple with dopamine-Cy5. Each antibody can couple with two PBA-G3 dendrimers loaded with Cy5 fluorescent molecules. In one embodiment, 6.67 μM antibodies that already react with PBA-G3 dendrimers is mixed with dopamine-Cy5 (the final concentration is 1.2 mM) to obtain complex as antibodies-dendrimers-Cy5. Then UV-vis spectrophotometer is used to measure the number of Cy5 that conjugates to every antibody. The experiment result shows that 31 Cy5 molecules conjugate to each antibody. In another embodiment, PBA-G2 dendrimers load Cy5 first, then they conjugate to antibodies.

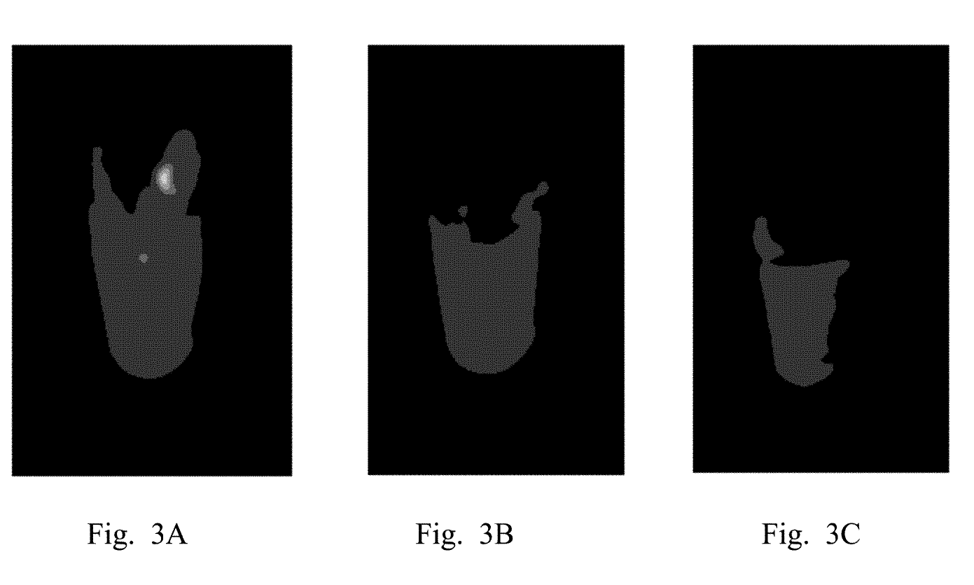

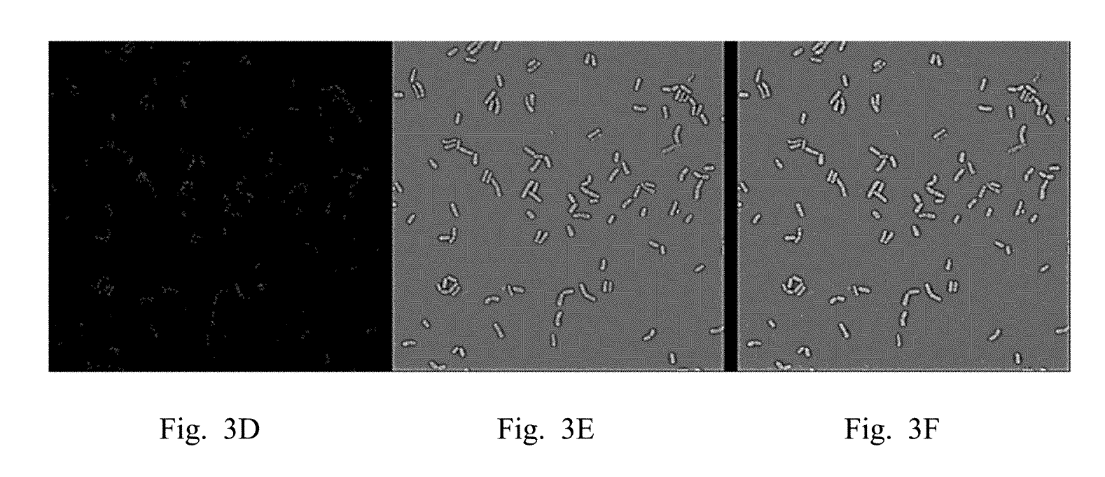

FIGS. 3A-3C are photos of fluorescent intensity of dendrimers loaded with Cy5 fluorescent molecules and modified by antibodies, in accordance with one embodiment of the present invention. The sample of FIG. 3A is the original solution without dilution. The sample of FIG. 3B is 10-fold dilution of the original solution. The sample of FIG. 3C is 100-fold dilution of the original solution. The sample remains comparable fluorescent intensity after 100-fold dilution. FIG. 3D is an immuno-staining image of Streptococcus pneumoniae labeled by dendrimers loaded with Cy5 fluorescent molecules and modified by antibodies, in accordance with one embodiment of the present invention. FIG. 3E is a bright-field image of FIG. 3D. FIG. 3F is a superimposed image of FIG. 3D and 3E. It is verifiable from the superimposed image that the method provided by the present invention has bio-specificity and biological recognition ability.

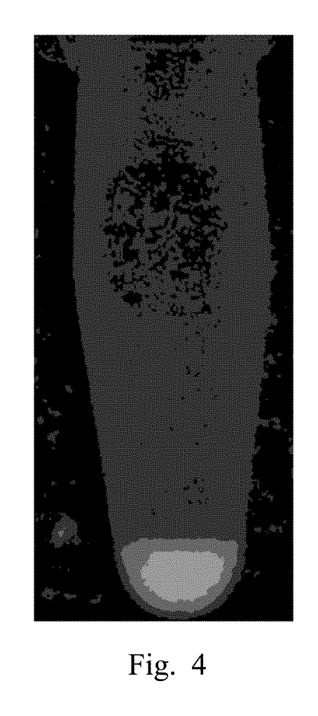

In some embodiments, PBA-G3 dendrimers can load anti-cancer drugs and couple with antibodies. In one embodiment, the anti-cancer drugs can be doxorubicin (DOX), which has self- fluorescein. One equivalent PBA-G3 dendrimer is mixed with twenty equivalents DOX in dimethyl sulfoxide (DMSO) solution. The solution is stirred vigorously at room temperature for 2 hour. Methanol is added into the solution and yield the pink precipitant as the product. Then, the product was collected by centrifugation to obtain the PBA-dendrimers loaded with DOX. FIG. 4 is a photo of fluorescent intensity of PBA-dendrimers loaded with DOX and modified by antibodies, in accordance with one embodiment of the present invention. The PBA-G3 dendrimers loaded with DOX is anchored on a Herceptin antibody, which can recognize breast cancer cell. The experiment result shows each PBA-G3 dendrimer can load about 30 DOX molecules in average. In another embodiment, PBA-G3 dendrimers load DOX first, then the PBA-G3 dendrimers loaded with DOX are anchored on a Herceptin antibody.

In some embodiments, PBA-dendrimers can load peptides. For example, dopamine-NCS reacts with peptides to form dopamine derivatives with peptides. The function for peptides can be therapeutic agents or recognition ability of antigen or bacteria. If the function is therapeutic agents, the implementation method will be the same as the embodiments related to DOX. If the function is recognition ability of antigen or bacteria, the PBA-dendrimers loaded with peptides will not need to couple with antibodies. Instead, three to five PBA terminal groups of the dendrimers couple with dopamine derivatives that have peptides. Other terminal groups can couple with drugs or fluorescent molecules. In one embodiment, PBA-G3 dendrimers can load peptides that can recognize epidermal growth factor receptor (EGFR). In lung cancer, breast cancer and colorectal cancer, cancer cells often have abnormal proliferation of EGFR, thus it will induce the activation of downstream transduction factors to cause cancer cells proliferate. Therefore, the effect of targeted-therapy can be achieved by PBA-dendrimers loaded with peptides that can recognize EGFR and drugs that can disconnect the signal transduction of EGFR.

In another embodiment, S7 peptides that can recognize Streptococcus pneumoniae are used. For example, one equivalent S7 peptides are mixed with one equivalent dopamine-NCS to obtain the dopamine derivative shown as Formula (IV). One equivalent PBA-G3 dendrimer reacts with four equivalents dopamine derivatives of Formula (IV) and twenty-eight equivalents dopamine-FITC to obtain dendrimers of which partial terminal groups are S7 peptides and fluorescent molecules. The dendrimers will not need to be modified by antibodies because the S7 peptides loaded on the dendrimers have recognition ability for Streptococcus pneumoniae.

In various embodiments of the present disclosure, a novel dendrimer of Formula (I) is provided. G0-G10 represent generation-0 to generation-10 dendrimers; the dendrimers comprise a plurality of branches; each branch comprises terminal groups; and n is a integer from 4-4096.

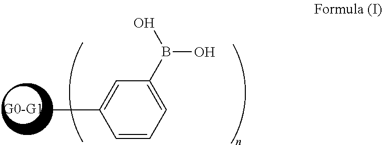

The advantage of the embodiments of the present disclosure is to provide biological modified dendrimers. The dendrimers can load drugs or fluorescent molecules efficiently,

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. An antibody comprising:
a polyamidoamine (PAMAM) dendrimer comprising a plurality of branches, and each of the branches having a phenylboronic acid (PBA) terminal group; and
a first functional groups bonded to at least one of the PBA terminal groups.

2. The antibody of claim 1, wherein the first functional group comprises a drug group, a fluorescent group, a peptide group, or a dopamine derivative group.

3. The antibody of claim 2, wherein the drug group comprises doxorubicin (DOX).

4. The antibody of claim 2, wherein the fluorescent group comprises a fluorescein isothiocyanate (PITC) group or a Cyanine dye group.

5. The antibody of claim 1, wherein the first functional group comprises a group of

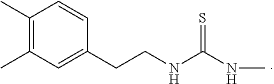

6. The antibody of claim 1, further comprising a second functional group, wherein the second functional group comprises a drug group or a fluorescent group, and the second functional group is different from the first functional group.
